(19) United States
(12) Patent Application Publication
(10) Pub. No.: US 2017/0332934 A1
(43) Pub. Date: Nov. 23, 2017

(54) NEUROCEREBRAL ASSESSMENT USING STIMULATED EEG RESPONSE

(71) Applicant: NIBS NEUROSCIENCE TECHNOLOGIES LTD., Zur Moshe (IL)

(72) Inventors: Iftach DOLEV, Zur Moshe (IL); Hilla FOGEL, Beit Dagan (IL)

(21) Appl. No.: 15/327,423

(22) PCT Filed: Jul. 28, 2015
(86) PCT No.: PCT/IL2015/050774

§ 371 Date: Jan. 19, 2017

Related U.S. Application Data
(60) Provisional application No. 62/030,276, filed on Jul. 29, 2014.

(51) Int. Cl. A61B 5/0484; A61B 5/00

(52) U.S. Cl. CPC A61B 5/0484; A61B 5/7246

(57) ABSTRACT

The present disclosure provides methods, systems and devices for assessing brain neural activity and/or characteristics by providing at least two TMS stimulation pulses, obtaining an EEG indicative of brain activity responses associated with the at least two TMS stimulation pulses and compare the brain activity responses to evaluate neural characteristic of the brain.

### TECHNICAL FIELD

The present disclosure generally relates to the field of neurocerebral and neural functionality assessment.

### BACKGROUND

Obtaining insights regarding brain activity and function has proven to be essential in multiple domains, such as assessment of cognitive abilities, detection/diagnostics of neuro-related conditions, behavioral research and others.

Current methods for obtaining such insights include brain imaging, mental status testing, physical and neurological exams, blood tests and the like. Some of these methods generally require an active engagement of the subject in performing mental actions and/or can detect only severe and late-stage conditions in the brain and in many of the current methods, the precision/accuracy is not always sufficient.

There is thus a need in the art for methods, devices and systems for assessing brain activity and function that enable accurate detection of early-stage conditions, without necessitating an active engagement of the subject in various mental tasks.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided herein devices, systems and methods for assessing a neurocerebral state and/or condition in a subject. According to some embodiments, the assessment is achieved by stimulating the brain and analyzing a brain response to the stimulation. According to some embodiments, the stimulation includes inducing a magnetic field/flux onto at least some areas of the brain. According to some embodiments, a brain response measurement is obtained using at least one electrode configured to measure activity of at least one area of the brain. According to some embodiments, measuring the brain activity includes detecting electric and/or electromagnetic activity of neuro-networks within the brain. According to some embodiments, the measuring of brain activity may include utilizing an electroencephalogram (“EEG”).

According to some embodiments, the stimulation is designed to instigate a response activity in the brain. According to some embodiments, the response activity is analyzed for assessing neurological/neurocerebral characteristics, functions, behaviors and/or various conditions.

According to some embodiments, stimulating the brain may include a plurality of stimuli. According to some embodiments, the plurality of stimuli may be temporally spaced therebetween and/or sequential. According to some embodiments, the plurality of stimuli is a stimulation burst. According to some embodiments, the analysis may include analyzing at least some brain responses resulting from at least some stimuli of the stimulation burst. According to some embodiments, assessing the neurocerebral state and/or condition in a subject is achieved by comparing the at least some brain responses with a set of expected/predefined brain responses of a normal brain and/or one or more brains having a neurological/neurocerebral condition.

Advantageously, such an assessment does not necessitate an active mental engagement of the subject, for example in solving mental puzzles or undertaking various cognitive tasks. Advantageously, such an assessment is objective and accurate, and may not be prone to the variations and inconsistencies associated with current methods of assessing brain activity and function.

According to some embodiments, the assessment may indicate various mental, neurological/neurocerebral conditions at an early-stage thereof. Advantageously, such an indication/detection at an early-stage may provide an opportunity to offer an effective treatment of the detected condition.

According to some embodiments, the stimulation and/or the measuring are non-invasive. Advantageously, the none-invasiveness may facilitate safety, convenience and even lower costs of the assessment and or detection of various conditions.

According to some embodiments, there is provided a method for assessing brain activity of a subject, the method including providing at least three electric stimulation pulses at a predetermined intensity to a region of the brain of a subject, detecting at least three respective electroencephalographic (EEG) signals, each signal obtained in response to each one of the at least three TMS pulses, comparing between at least two EEG signals obtained in response to at least two TMS pulses, and assessing brain activity of the subject at least based on the comparison.

According to some embodiments, the electric stimulation pulses include magnetically induced electric stimulation pulses.

According to some embodiments, the magnetically induced electric stimulation pulses include Transcranial Magnetic Stimulation (TMS) pulses.

According to some embodiments, the electric stimulation pulses are electrically induced electric stimulation pulses.

According to some embodiments, the brain activity includes one or more brain neural properties.

According to some embodiments, the one or more brain neural properties includes excitability, plasticity or both.

According to some embodiments, the predetermined intensity is based on a motor threshold intensity associated with the subject.

According to some embodiments, the method further includes establishing a motor threshold intensity associated with the subject including providing multiple TMS pulses at varying intensities to a motor-associated region of the brain, monitoring a motor-reaction of the subject, and detecting an intensity, above which a motor-reaction is observed.

According to some embodiments, the predetermined intensity is more than 80% of the motor threshold intensity.

According to some embodiments, the predetermined intensity is less than 120% of the motor threshold intensity.

According to some embodiments, the method further includes providing a first TMS pulse to a first region of the brain of a subject, detecting a first EEG signal obtained in response to the first TMS pulse, providing a second TMS pulse to the first region of the brain of a subject, detecting a second EEG signal obtained in response to the first TMS pulse, providing a third TMS pulse to the first region of the brain of a subject, detecting a third EEG signal obtained in response to the first TMS pulse, comparing between at least two of the first, second and third EEG signals, and assessing brain activity of the subject at least based on the comparison.

According to some embodiments, the method further includes providing at least three TMS pulses to a second region of the brain of a subject, detecting at least three EEG signals, each signal obtained in response to each one of the at least three TMS pulses, comparing between at least two EEG signals obtained in response to at least two TMS pulses, and assessing brain activity of the subject based on the comparison between at least two EEG signals and on a comparison between EEG signals obtained in response to TMS pulses provided to the first region and EEG signals obtained in response to TMS pulses provided to the second region.

According to some embodiments, the first and second regions include the right and left brain hemispheres, respectively.

According to some embodiments, the comparison includes comparing between amplitudes of characterized EEG distinct temporal signatures or initial EEG responses to TMS, slopes (rise time) of either characterized EEG distinct temporal signatures or initial EEG responses to TMS, area under curve (AUC) of TMS responses either entire AUC or of a single priority targeted characterized EEG distinct signatures, and/or any combination thereof.

According to some embodiments, there is provided a system for assessing brain activity of a subject, the system including an electric stimulation device configured to provide electric stimulation pulses to a region of the brain of a subject, an electroencephalographic (EEG) device configured to monitor neural activity in the brain of the subject and provide EEG signals indicative thereof, and processing circuitry configured to compare between at least two EEG signals obtained in response to at least two electric stimulation pulses and to assess brain activity of the subject at least based on the comparison.

According to some embodiments, the electric stimulation device includes a transcranial magnetic stimulation (TMS) device and the electric stimulation pulses include TMS pulses.

According to some embodiments, the brain activity includes one or more brain neural properties.

According to some embodiments, the one or more brain neural properties includes excitability, plasticity or both.

According to some embodiments, the TMS device is configured to provide at least one TMS stimulation burst including a plurality of TMS stimulation pulses, wherein the plurality of TMS stimulation pulses are temporally separated.

According to some embodiments, the processing circuitry is configured to compare between at least two EEG signals obtained in response to at least two TMS pulses of the TMS stimulation burst and to assess brain activity of the subject at least based on the comparison.

According to some embodiments, the TMS device is further configured to: provide at least one TMS stimulation session including multiple TMS stimulation bursts, wherein the TMS stimulation bursts are temporally separated by inter burst intervals.

According to some embodiments, the processing circuitry is configured to obtain EEG signals indicative of brain activity response associated with at least some of the TMS stimulation bursts, and to generate a representative EEG burst activity response including representative EEG pulse activity responses.

According to some embodiments, the inter burst intervals have a duration of at least 20 seconds.

According to some embodiments, a representative EEG burst includes an average of EEG signals indicative of brain activity response associated with at least some of the TMS stimulation bursts.

According to some embodiments, the processing circuitry is further configured to compare between at least two representative EEG pulse activity responses and to assess brain activity of the subject at least based on the comparison.

According to some embodiments, the processing circuitry is configured to perform the comparison based at least on amplitude of characterized EEG distinct temporal signature or initial EEG response to TMS, slope (rise time) of either characterized EEG distinct temporal signature or initial EEG response to TMS, area under curve (AUC) of each TMS response (charge transfer) either entire AUC or of a single priority targeted characterized EEG distinct signature, or any combination thereof.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a neurocerebral assessment setting, according to some embodiments;

FIG. 2 schematically illustrates a stimulation burst, according to some embodiments;

FIG. 3 schematically illustrates a stimulation session, according to some embodiments;

FIG. 4 schematically illustrates an EEG response to a stimulation burst in a healthy brain, according to some embodiments;

FIG. 5 schematically illustrates a stimulation intensity operation range, according to some embodiments;

FIG. 6 schematically illustrates a method for establishing a motor threshold value, according to some embodiments;

FIG. 7 schematically illustrates a method for providing stimulated EEG responses burst, according to some embodiments; and

FIG. 8 schematically illustrates a method for providing stimulated EEG responses session, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The brain serves as the center of the nervous system in most invertebrate and all vertebrate animals, and is the most complex organ in the body of vertebrate animals, comprising billions of neurons each of which is connected by synapses to thousands of other neurons to perform mental tasks vital to the functioning and wellbeing of the animal. In human beings, the cognitive abilities of our brains play a major role in defining one's role, contribution and wellbeing within a society and individually. The neural system of the brain is referred to as the neurocerebral system.

Unfortunately, our brains are prone to multiple conditions which may impair the ability thereof to perform mental tasks. Some of these conditions include neurodegenerative conditions that may affect the structure or function/characteristics of the neurons of the brain and even may include death of the neurons. Several conditions that fall within the category of neurodegenerative conditions include Parkinson's, ALS, Alzheimer's, Huntington's and others. Other conditions that might affect the brain may include chronic pain, ADD/ADHD, other types of dementia besides Alzheimer's disease and many others that affect our ability to perform regular tasks in our daily life, and some may even impose intolerable/insufferable disorders.

Despite the severity of the conditions that might affect the brain, the detection/diagnostics of these conditions usually occurs after the condition has reached a late stage and has begun to noticeably affect the function of the human being. Unfortunately, the effectiveness of a potential treatment is compromised due to the delayed/late stage of the condition.

According to some embodiments, there are provided systems, devices and methods for assessing neurocerebral functioning and/or characteristics. According to some embodiments, the assessment may be conducted by inducing a stimulus to the brain, and measuring the reactive activity of the brain (or neural networks therein) to the stimulus. According to some embodiments, the assessment may be conducted by inducing a plurality of stimuli to the brain, and measuring the reactive activity of the brain (or neural structures therein) to the stimuli.

According to some embodiments, the reactive activity of the brain may be indicative of some characteristics of the neural structures and/or neural network function within the brain, and these characteristics may facilitate an assessment of the neurocerebral functioning and/or characteristics, thereby facilitating the detection/diagnostics of various conditions affecting the brain.

According to some embodiments, such an assessment may be indicative of a brain/neural condition even before noticeable behavioral/functional effects are perceived by the subjects and/or observers thereof. Advantageously, the ability to detect conditions even before apparent effects may facilitate an early stage detection of the condition, and potentially assist in the effectiveness of the treatment.

According to some embodiments, the devices, systems and/or methods may be used for assessing the progress and/or state of a diagnosed subject. According to some embodiments, the devices, systems and/or methods may be used for measuring a stage/severity of a disorder or condition. According to some embodiments, the devices, systems and/or methods may be used for detecting and/or assessing neural network abnormalities.

According to some embodiments, such an assessment may provide precise indication of specific conditions, and thereby mitigate the risk of misdiagnosis and the undesired complications related thereto.

According to some embodiments, the stimulation is an electric stimulation. According to some embodiments, the stimulation is an electrically induced electric stimulation. According to some embodiments, the stimulation is a magnetically induced electric stimulation. According to some embodiments, the stimulation is a Transcranial-Magnetic-stimulation.

According to some embodiments, the stimulation includes inducing a magnetic flux/field to the brain or portions thereof. According to some embodiments, the magnetic stimulation may include a Transcranial-Magnetic-stimulation (TMS). According to some embodiments, the stimulation may include multiple TMS magnetic pulses at determined intensities. According to some embodiments, the TMS intensity may refer to the flux of the induced magnetic field. According to some embodiments, the TMS intensity may be measured/presented in Tesla units. According to some embodiments, the TMS intensity may be measured/presented as a percentage of a reference value.

According to some embodiments, measuring the reactive activity may be facilitated by measuring an electric/electromagnetic activity of neural structures in the brain. According to some embodiments, measuring the reactive activity may be facilitated by utilizing at least one electrode, configured to measure variations of electric/electromagnetic fields indicative of an activity of specific neural networks in the brain. According to some embodiments, measuring the reactive activity may be facilitated by utilizing an EEG device/system.

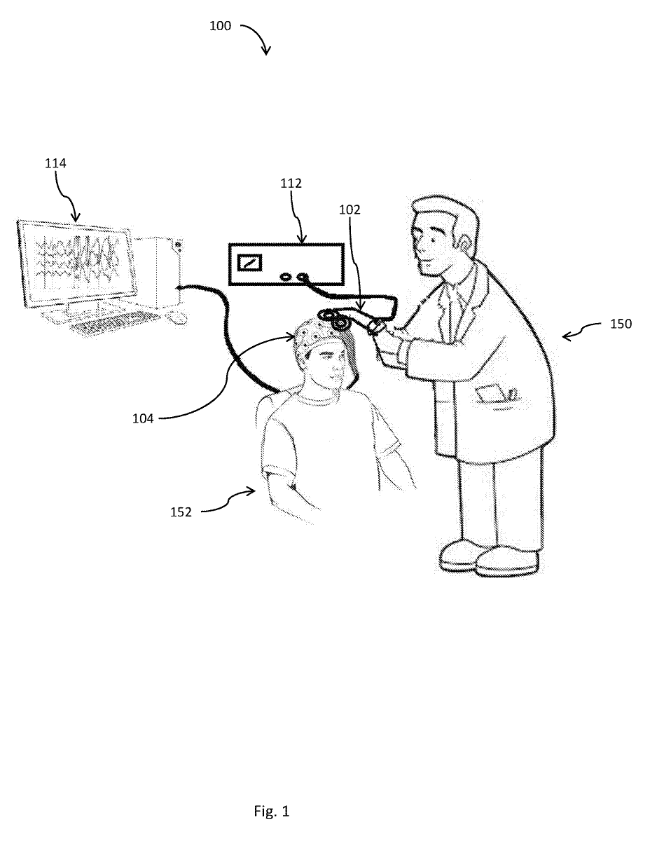

Reference is now made to FIG. 1, which schematically illustrates a setting 100 of stimuli-reaction neural function assessment of a subject 152 by a caregiver 150, according to some embodiments. As illustrated, caregiver 150 places a stimulating device, such as but not restricted to TMS-inducer 102, at a certain position on/near the head of subject 152. TMS-inducer 102 is provided with control signals and electric energy from a controller, such as a TMS-controller 112. TMS-controller 112 is configurable for enabling a controllable stimulation by TMS-unit 112. According to some embodiments, a controllable stimulation may include control over the intensity, duration, frequency, flux and/or other stimulation-related parameters and patterns of stimulation.

At least some brain activity sensors, such as EEG-electrodes 104, are placed on the head of subject 152, and configured to measure neural activity of the brain of subject 152 of defined areas thereof. According to some embodiments, EEG-electrodes 104 are in communicational link with an analyzer/controller, such as an EEG-analyzer 114, configured to obtain EEG signals from EEG-electrodes 104.

According to some embodiments, EEG-analyzer 114, or other processing circuitry (such as remote servers, cloud computing service, a local computer, and the like), is configured to analyze the obtained EEG signals and detect reactive activity associated with induced electrical stimulation, such as TMS stimulations induced by TMS-inducer 102. According to some embodiments, EEG-analyzer 114 is configured to detect neural characteristics of the brain of subject 152 (or neural structures thereof) by measuring the intensities of the reactive activity, and comparing the intensities with model/reference activities expected assuming various conditions and/or lack thereof. According to some embodiments, EEG-analyzer 114 is configured to detect neural network activity characteristics of the brain of subject 152 (or neural structures thereof) by measuring the intensities of the reactive activity and comparing the ratios between various pairs therefrom, and comparing the intensities with model/reference activities expected assuming various conditions and/or lack thereof.

According to some embodiments, EEG-analyzer 114 is configured to analyze the EEG signals relying on their amplitudes, slopes, frequencies, delays, area under curver and the like. According to some embodiments, EEG-analyzer 114 is configured to perform a frequency analysis of the EEG signals. According to some embodiments, EEG-analyzer 114 is configured to perform noise reduction filtration on the EEG-signals. According to some embodiments, EEG-analyzer 114 is configured to perform noise cancelation filtration on the EEG-signals. According to some embodiments, EEG-analyzer 114 is configured to perform a temporal analysis of the EEG signals.

According to some embodiments, Neuronal stimulator, such as, TMS-unit 102 is configured to induce multiple stimuli pulses. According to some embodiments, the multiple stimuli pulses are substantively equal in intensity. According to some embodiments, the multiple stimuli pulses are substantively equal in duration. According to some embodiments, the multiple stimuli pulses are substantively similar in slopes. According to some embodiments, the multiple stimuli differ in intensity, duration, and/or slopes.

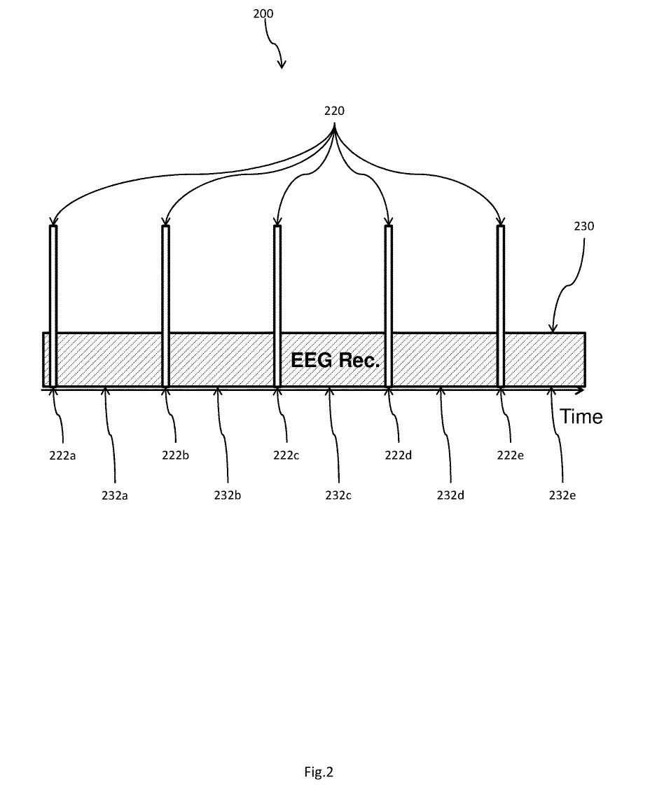

Reference is now made to FIG. 2, which schematically illustrates a stimulation burst 200, according to some embodiments. Stimulation burst 200 may include multiple stimulation events, such as stimuli pulses 220, configured to induce stimulation, evoked by stimulator device, such as TMS, at determined times and intensities. According to some embodiments, stimulation burst 200 may further include an EEG-monitor 230 for measuring the response activity to stimuli pulses 220. According to some embodiments, EEG-monitor 230 may be continuous throughout the period of stimulation burst 200. According to some embodiments, EEG monitor 230 may be intermittent.

According to some embodiments, stimuli pulses 220 comprise a first stimulus pulse 222a followed by a first pulse delay interval 232a, a second stimulus pulse 222b followed by a second pulse delay interval 232b, a third stimulus pulse 222c followed by a third pulse delay interval 232c, a fourth stimulus pulse 222d followed by a fourth pulse delay interval 232d and a fifth stimulus pulse 222e followed by a fifth pulse delay interval 232e.

According to some embodiments, pulse delay intervals 232a, 232b, 232c, 232d, 232e are configured to enable distinguishing between EEG recording/monitoring of reaction activity associated with each of stimulus pulses 222a, 222b, 222c, 222d, 222e.

According to some embodiments, stimulus pulses 222a, 222b, 222c, 222d, 222e may be substantively equal in intensity. According to some embodiments, stimulus pulses 222a, 222b, 222c, 222d, 222e may vary in intensity.

According to some embodiments, stimulus pulses 222a, 222b, 222c, 222d, 222e may be substantively equal in duration. According to some embodiments, stimulus pulses 222a, 222b, 222c, 222d, 222e may vary in duration.

According to some embodiments, pulse delay intervals 232a, 232b, 232c, 232d, 232e may be substantively equal in duration. According to some embodiments, pulse delay intervals 232a, 232b, 232c, 232d, 232e may vary in duration.

According to some embodiments, the duration of a pulse is in the range of 0.5 ms to 2 ms. According to some embodiments, the duration of a pulse is in the range of 1 ms to 1.5 ms. According to some embodiments, the duration of a pulse is approximately 1 ms.

According to some embodiments, the delay intervals are in the range of 100 ms to 2 s. According to some embodiments, the delay intervals are in the range of 250 ms to 1 s. According to some embodiments, the delay intervals are approximately of 500 ms. According to some embodiments, the pulse frequency within a burst is in the range of 0.1 Hz to 10 Hz. According to some embodiments, the pulse frequency within a burst is in the range of 1 Hz to 5 Hz. According to some embodiments, the pulse frequency is approximately 2 Hz.

According to some embodiments, the reaction activity of the neural network in the brain is expected to vary between consecutive stimulus pulses. This may be attributed to the neuro-structural characters of plasticity and excitability. As a result, the reaction activity associated with the second stimuli pulse may be considerably higher than the reaction activity associated with the first stimuli pulse. Additionally, in later stimulus pulses, a gradual decrease in the reaction activity may be expected.

The amount/ratio of increased and/or decreased activity may be indicative of the characteristics of the neuronal network (neural structures), such as plasticity and excitation and inhibition. Consequently, one may detect irregularities in these characteristics and associate them with various conditions that may lead to changes in these characteristics.

According to some embodiments, a stimulation burst may include 1 or more stimuli pulses. According to some embodiments, a stimulation burst may include 2 or more stimuli pulses. According to some embodiments, a stimulation burst may include 3 or more stimuli pulses. According to some embodiments, a stimulation burst may include 4 or more stimuli pulses. According to some embodiments, a stimulation burst may include 5 or more stimuli pulses. According to some embodiments, a stimulation burst may include 2 to 10 stimuli pulses. According to some embodiments, a stimulation burst may include 10 or more stimuli pulses.

According to some embodiments, multiple stimulation bursts may be induced to generate a stimulation session. According to some embodiments, the bursts are substantively similar there between. According to some embodiments, the bursts are temporally separated with relaxation intervals/periods in between (inter burst interval). According to some embodiments, the inter burst interval (relaxation intervals/periods) are configured to eliminate reaction effects (“memory”) between different bursts. According to some embodiments, the reaction activity associated with at least some of the bursts facilitated noise-reduction and/or elimination of inconsistent/outstanding measurements.

As used herein, the term “inter burst interval” may be interchangeable with the term “relaxation period” and may refer to a period of time between bursts that is configured to facilitate separation and/or effect mitigation between different stimulation bursts.

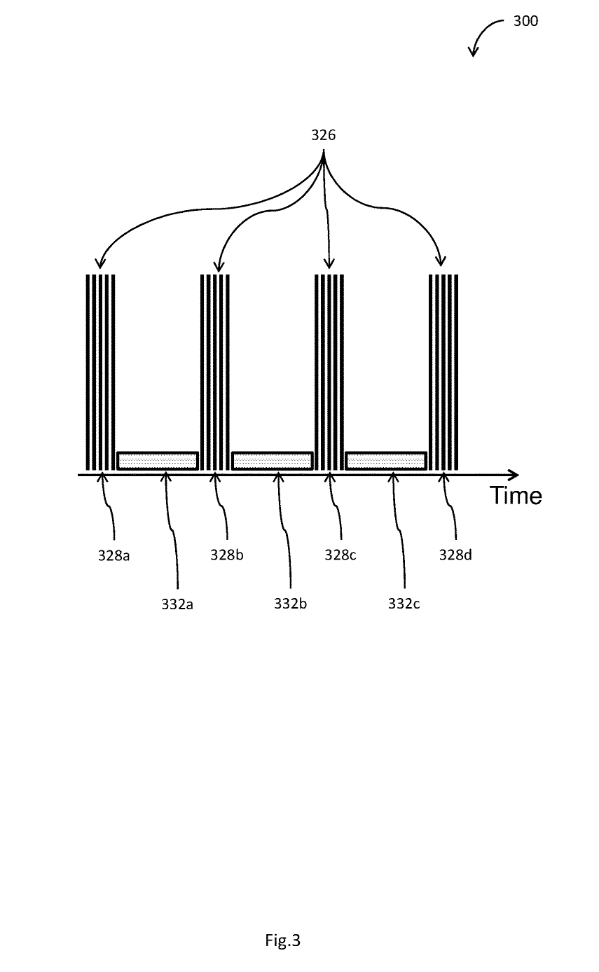

Reference is now made to FIG. 3, which schematically illustrates a stimulation session 300, according to some embodiments. Stimulation session may include multiple stimulation bursts 326. According to some embodiments, stimulation bursts may be temporally spaced apart, for example a first stimulation burst 328a is followed by a first wait period 332a, a second stimulation burst 328b is followed by a second wait period 332b, a third stimulation burst 328c is followed by a third wait period 332c which is followed by a fourth stimulation burst 328d.

According to some embodiments, each of wait periods 332a, 332b and 332c is configured to mitigate/eliminate a considerable portion or any effect that may occur between the reaction activity associated with stimulation bursts (inter burst interval) 382a, 382b, 382c and 382d.

According to some embodiments, the term inter burst interval may be interchangeable with the term “wait period”, “relaxation period” or “buffer period”.

According to some embodiments, due to the inter burst interval periods, the reaction activity associated with each of the bursts may be expected to be similar. Therefore, any outstanding reaction activity associated with a certain burst that doesn't match or resemble reaction activity associated with other bursts may be considered false or may be neglected and not considered.

According to some embodiments, reaction activity associates with at least some of the bursts are compared for noise reduction, averaging, statistical analysis and the like.

According to some embodiments, inter burst intervals 332a, 332b and 332c may have a substantively similar duration. According to some embodiments, wait periods 332a, 332b and 332c may have different durations.

According to some embodiments, an inter burst interval period may be at least 20 seconds. According to some embodiments, an inter burst interval period may be at least 30 seconds. According to some embodiments, a inter burst interval period may be at least 40 seconds. According to some embodiments, an inter burst interval period may be at least 50 seconds. According to some embodiments, an inter burst interval period may be at least 60 seconds. According to some embodiments, an inter burst interval period may range from 10 seconds to 20 minutes. According to some embodiments, an inter burst interval period may range from 20 seconds to 10 minutes. According to some embodiments, an inter burst interval may range from 30 seconds to 50 minutes.

According to some embodiments, a stimulation session may include 2 or more simulation bursts. According to some embodiments, a stimulation session may include 3 or more stimulation bursts. According to some embodiments, a stimulation session may include 5 to 10 stimulation bursts. According to some embodiments, a stimulation session may include 10 or more stimulation bursts.

According to some embodiments, a stimulation burst may be essentially as described in FIG. 2.

According to some embodiments, some parameters, characteristics and/or features that may be considered for assessing the neural network characteristics may include one or more of the following: amplitudes, slopes, frequencies, delays, area under curve and the ratios between. According to some embodiments, these parameters may be in response to stimulation pulses, or with association thereto.

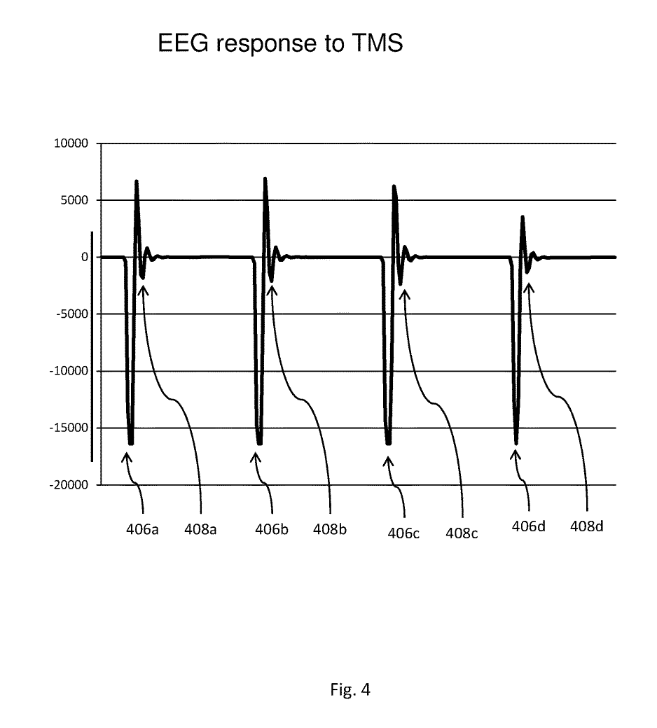

Reference is now made to FIG. 4, which schematically illustrates an EEG response activity to a stimulation burst in a normal brain, according to some embodiments. As illustrated, the stimulation burst includes four stimulation pulses, the EEG recording thereof is illustrated in a first pulse recording 406a, a second pulse recording 406b, a third pulse recording 406c and a fourth pulse recording 406d. Following each pulse recording, there is a response recording, namely a first response recording 408a, a second response recording 408b, a third response recording 408c and a fourth response recording 408d. As illustrated, second response recording 408b and third response recording 408c are greater than first response recording 408a, which may be attributed to the excitability or plasticity characteristics of the relevant neural network, while forth response recording 408d is considerably reduced compared to the rest of the responses recorded, which may be attributed to the adaptation characteristics of the relevant neural network.

According to some embodiments, responses that vary from the normal response or a normal response range may indicate various abnormal neural network characteristics.

According to some embodiments, the intensity of pulses is determined as a percentage of a reference value. According to some embodiments, the reference value is determined as a percentage from the personal determined motor-threshold value.

According to some embodiments, a motor threshold is a stimulation intensity at which a motoric reaction may be triggered and/or detected as a consequence thereto.

According to some embodiments, a motor threshold value is established by increasingly incrementing stimulation intensity, until a motoric reaction is detected.

According to some embodiments, the motoric threshold may vary from one person to another. According to some embodiments, using a TMS stimulator to induce focal electrical stimulation in the brain, the motor threshold value may be in the range of 1.5 to 2.5 Tesla. According to some embodiments, the motor threshold value may be in the range of 1.7 to 2.3 Tesla. According to some embodiments, the motor threshold value may be in the range of 1.8 to 2.2 Tesla. According to some embodiments, the motor threshold value may be in the range of 1.9 to 2.1 Tesla. According to some embodiments, the motor threshold value may be in the range of 48% to 52% of the maximal TMS device intensity. According to some embodiments, the motor threshold value may be approximately 50% of the maximal TMS device intensity.

Once a motor threshold value is established for a certain person, one may evaluate/calculate an “operational range”, which is a range of intensity values in which a reaction activity may be observed and activity corresponds to the intensity of the stimuli. According to some embodiments, the term “operational range” may be interchangeable with the terms “relevant range” or “active range”.

According to some embodiments, an “operational range” ranges from 60% to 140% of the motor threshold. According to some embodiments, an “operational range” ranges from 80% to 120% of the motor threshold. According to some embodiments, an “operational range” ranges from 50% to 150% of the motor threshold.

According to some embodiments, the intensity of the stimuli pulses may be a value within the “operational range”. According to some embodiments, the intensity of the stimuli pulses may be referred to as an “operational point”.

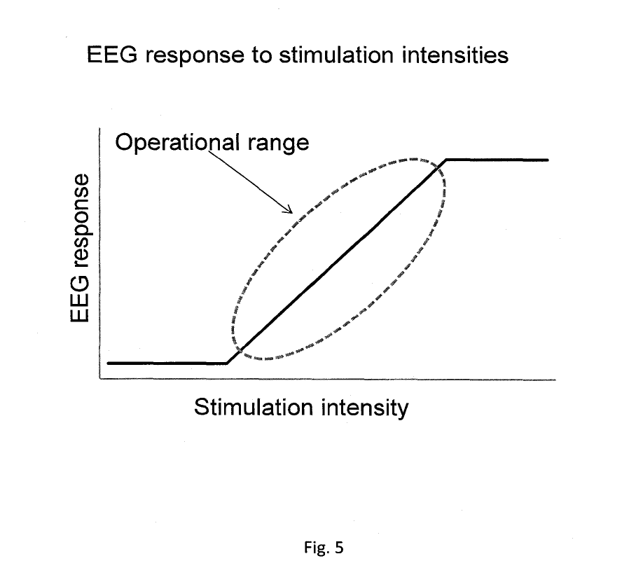

Reference is now made to FIG. 5, which schematically illustrates a stimulation intensity operational range, according to some embodiments. As illustrated, the EEG response to low stimulation intensities is relatively unchanged, until the stimulation intensity surpasses a certain value (lower threshold), then the EEG response reacts/increases as the stimulation intensity increases, until the stimulation intensity reaches another value (upper threshold) in which the EEG response no longer responds to increases in the stimulation intensity.

According to some embodiments, the operational range is a range of intensity values between the lower threshold and the upper threshold.

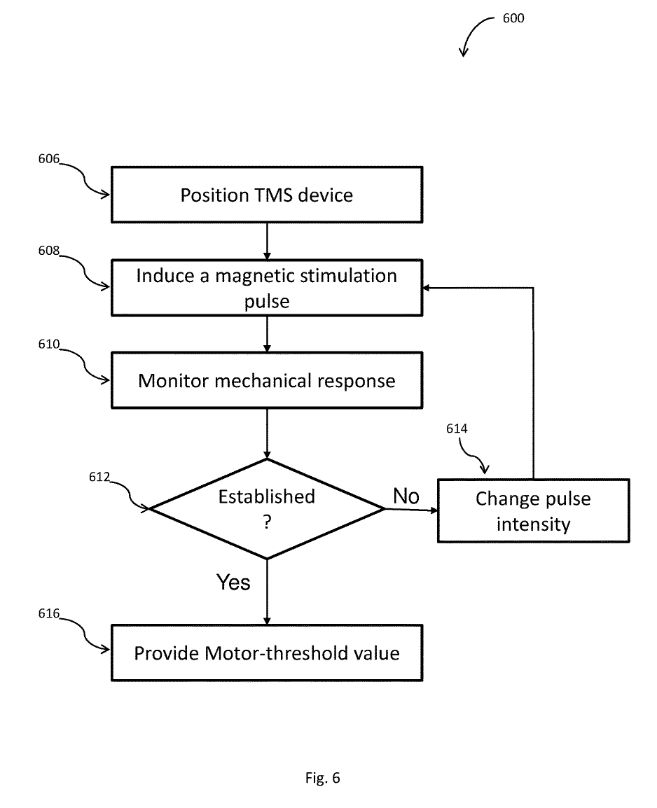

Reference is now made to FIG. 6, which schematically illustrates a method 600 for establishing a motor threshold value, according to some embodiments. According to some embodiments, the TMS device/unit is positioned (step 606) in a determined position on/near the head of the subject, then a magnetic stimulation pulse is induced (step 608), then the mechanical response is monitored (step 610) for detecting triggered motoric movement. According to some embodiments, if a motoric movement is detected, and a motor threshold established (step 612), the motor threshold value is provided (step 616), otherwise, the pulse intensity is changed (step 614) and we iterate the steps from inducing the magnetic stimulation pulse (step 608).

According to some embodiments, changes to the pulse intensity (step 614) include increasing the pulse intensity.

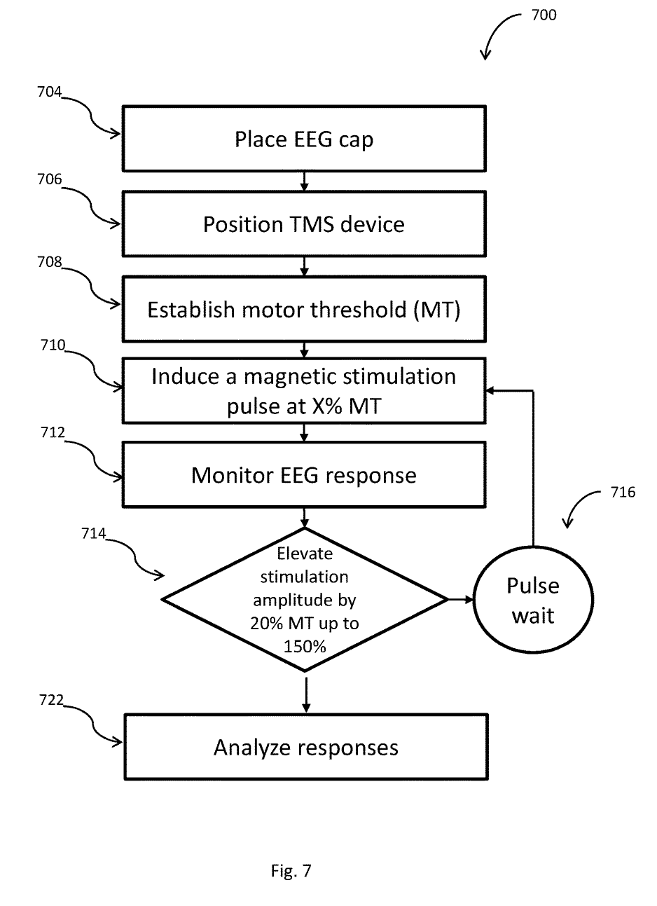

Reference is now made to FIG. 7, which schematically illustrates a method 700 for providing a stimulated EEG response session, according to some embodiments. According to some embodiments, an EEG cap is placed on the head of a subject (step 704), then a TMP device is positioned on/near the head of the subject (step 706), then a motor threshold (“MT”) is established (step 708), then a magnetic stimulation pulse is induced (step 710) having an intensity of X % of the established MT. According to some embodiments, the X % is a predetermined percentage of the MT. According to an operational range/value based on the motor threshold, then the EEG response activity is monitored, (step 712), then optionally steps 710 and 712 are repeated as the stimulation amplitude is elevated by a predetermined percentage up to a certain level, for example 20% up to 150%, (step 714) with a “pulse-wait” delay between iterations (step 716), and the response activity is analyzed (step 722).

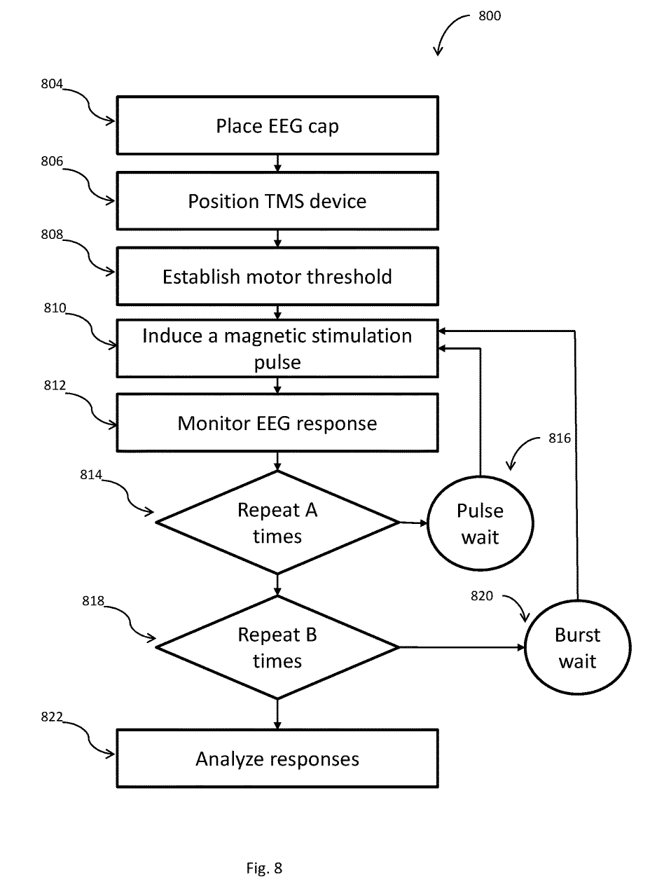

Reference is now made to FIG. 8, which schematically illustrates a method 800 for providing a stimulated EEG response session, according to some embodiments. According to some embodiments, an EEG cap is placed on the head of a subject (step 804), then a TMP device is positioned on/near the head of the subject (step 806), then a motor threshold is established (step 808), then a magnetic stimulation pulse is induced (step 810) according to an operational range/value based on the motor threshold, then the EEG response activity is monitored, (step 812), then optionally steps 810 and 812 are repeated A-number of times (step 814) with a “pulse-wait” delay between iterations (step 816), then optionally steps 810 to 814 are repeated B-number of times (step 818) with a “burst wait” (relaxation) delay between iterations (step 820) and the response activity is analyzed (step 822).

According to some embodiments, a stimulation threshold value is established. According to some embodiments, a stimulation threshold value is a TMS stimulation intensity value, above-which reactive neural activity may be observed/detected.

According to some embodiments, the TMS stimulation intensity is a “supra-threshold”, selected to be of a higher intensity than the stimulation threshold.

According to some embodiments, the TMS stimulation intensity is a “sub-threshold”, selected to be of a lower intensity than the stimulation threshold.

According to some embodiments, the analysis is conducted after the stimulation session. According to some embodiments, the analysis is conducted during the stimulation session.

According to some embodiments, A and B are predetermined numbers/values.

As used herein, the terms “neuron”, “neuro-structure”, “neural network” may be interchangeable.

As used herein, the term “TMS” may refer to Transcranial magnetic stimulation, which is a non-invasive method used to stimulate regions of the brain. In TMS, a magnetic field generator, such as a coil or an electromagnet, is placed near/on the head of the subject receiving the stimulation, an electric current is conducted through the coil and a magnetic flux gradient is induced as a result of the change in current through the coil. According to some embodiments, the coil (or electromagnet coil) is connected to a pulse generator, or controller, or stimulator configured to deliver electric current to the coil.

As used herein, the term “EEG” may refer to Electroencephalography, which is typically a non-invasive method for recording electrical activity of the brain along the scalp. An EEG measures voltage fluctuation resulting from ionic current within the neurons/neuro-structures of the brain. According to some embodiments, an EEG may refer to the recording of the brain's spontaneous and/or stimulated electrical activity over a period of time.

As used herein, the term “plasticity” may refer to neuroplasticity or brain plasticity which may encompass synaptic and/or non-synaptic plasticity, and may refer to changes in neural pathways and synapses and/or structure due to changes in behavior, environment, neural processes, thinking, emotions, injuries and stimulation.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-26. (canceled)

27. A method for assessing brain activity of a subject, the method comprising:
providing at least three electric stimulation pulses at a predetermined intensity to a region of the brain of a subject;
detecting at least three respective electroencephalographic (EEG) signals, each signal obtained in response to each one of said at least three TMS pulses;
comparing between at least two EEG signals obtained in response to at least two TMS pulses; and
assessing brain activity of the subject at least based on the comparison.

28. The method of claim 27, wherein the electric stimulation pulses comprise electrically induced electric stimulation pulses.

29. The method of claim 27, wherein the brain activity comprises one or more brain neural properties; wherein the one or more brain neural properties comprises excitability, plasticity or both.

30. The method of claim 27, wherein the predetermined intensity is based on a motor threshold intensity associated with the subject.

31. The method of claim 30, further comprising establishing a motor threshold intensity associated with the subject comprising:
providing multiple TMS pulses at a varying intensities to a motor-associated region of the brain;
monitoring a motor-reaction of the subject; and
detecting an intensity, above which a motor-reaction is observed.

32. The method of claim 31, wherein the predetermined intensity is more than 80% of the motor threshold intensity; and/or wherein the predetermined intensity is less than 120% of the motor threshold intensity.

33. The method of claim 27, further comprising:
providing a first TMS pulse to a first region of the brain of a subject;
detecting a first EEG signal obtained in response to the first TMS pulse;
providing a second TMS pulse to the first region of the brain of a subject;
detecting a second EEG signal obtained in response to the first TMS pulse;
providing a third TMS pulse to the first region of the brain of a subject;
detecting a third EEG signal obtained in response to the first TMS pulse;
comparing between at least two of the first, second and third EEG signals; and
assessing brain activity of the subject at least based on the comparison.

34. The method of claim 27, further comprising:
providing at least three TMS pulses to a second region of the brain of a subject;
detecting at least three EEG signals, each signal obtained in response to each one of said at least three TMS pulses;
comparing between at least two EEG signals obtained in response to at least two TMS pulses; and
assessing brain activity of the subject based on the comparison between at least two EEG signals and on a comparison between EEG signals obtained in response to TMS pulses provided to the first region and EEG signals obtained in response to TMS pulses provided to the second region.

35. The method of claim 34, wherein the first and second regions comprise the right and left brain hemispheres, respectively.

36. The method of claim 27, wherein the comparison comprises comparing between:
amplitudes of characterized EEG distinct temporal signatures or initial EEG responses to TMS,
slopes (rise time) of either characterized EEG distinct temporal signatures or initial EEG responses to TMS,
area under curve (AUC) of TMS responses either entire AUC or of a single priority targeted characterized EEG distinct signatures,
or any combination thereof.

37. A system for assessing brain activity of a subject, the system comprising:
an electric stimulation device configured to provide electric stimulation pulses to a region of the brain of a subject;
an electroencephalographic (EEG) device configured to monitor neural activity in the brain of the subject and provide EEG signals indicative thereof; and
processing circuitry configured to compare between at least two EEG signals obtained in response to at least two electric stimulation pulses and to assess brain activity of the subject at least based on the comparison.

38. The system of claim 37, wherein said electric stimulation device comprises a transcranial magnetic stimulation (TMS) device and the electric stimulation pulses comprise TMS pulses.

39. The system of claim 38, wherein the brain activity comprises one or more brain neural properties; wherein the one or more brain neural properties comprises excitability, plasticity or both.

40. The system of claim 38, wherein the TMS device is configured to:
provide at least one TMS stimulation burst comprising a plurality of TMS stimulation pulses,
wherein the plurality of TMS stimulation pulses are temporally separated.

41. The system of claim 40, wherein said processing circuitry is configured to compare between at least two EEG signals obtained in response to at least two TMS pulses of the TMS stimulation burst and to assess brain activity of the subject at least based on the comparison.

42. The system of claim 40, wherein said TMS device is further configured to:
provide at least one TMS stimulation session comprising multiple TMS stimulation bursts, wherein the TMS stimulation bursts are temporally separated by inter burst intervals.

43. The system of claim 42, wherein said processing circuitry is configured to obtain EEG signals indicative of brain activity response associated with at least some of the TMS stimulation bursts, and to generate a representative EEG burst activity response comprising representative EEG pulse activity responses.

44. The system of claim 42, wherein a representative EEG burst comprises an average of EEG signals indicative of brain activity response associated with at least some of the TMS stimulation bursts.

45. The system of claim 42, wherein said processing circuitry is further configured to compare between at least two representative EEG pulse activity responses and to assess brain activity of the subject at least based on the comparison.

46. The system of claim 38, wherein said processing circuitry is configured to perform said comparison based at least on:
amplitude of characterized EEG distinct temporal signature or initial EEG response to TMS,
slope (rise time) of either characterized EEG distinct temporal signature or initial EEG response to TMS,
area under curve (AUC) of each TMS response (charge transfer) either entire AUC or of a single priority targeted characterized EEG distinct signature, or any combination thereof.
